(19) United States
(12) Patent Application Publication
(10) Pub. No.: US 2003/0110524 A1
(43) Pub. Date: Jun. 12, 2003

(54) TRANSGENIC ORGANISMS AND THEIR USES

(76) Inventor: BRADLEY MICHAEL JOHN STRINGER, CARDIFF (GB)

Correspondence Address: David A. Jackson, KLAUBER & JACKSON, 411 Hackensack Avenue, Hackensack, NJ, 07601, US

(21) Appl. No.: 09/460,559

(22) Filed: Dec. 14, 1999

Related U.S. Application Data
(63) Continuation of application No. 08/836,077, filed on Jul. 28, 1997, now abandoned.

(51) Int. Cl. A01K 67/027; C12N 5/06; C12N 15/85

(52) U.S. Cl. 800/13; 800/14; 800/18; 800/25; 435/325; 435/455; 435/463

(57) ABSTRACT

Transgenic organisms (in particular, transgenic animals or plants) bearing positive and/or negative selectable markers are described along with their use in various methods including tissue/cell culture techniques, methods of making monoclonal antibodies, methods of selectively eliminating or depleting a particular tissue/cell type, methods of screening compounds for pharmacological activity and methods of determining the effect of a deficit in a first class of cells or the characteristics of a second class of cells in an organism.

The present invention relates to transgenic organisms having cells with one or more selectable phenotypes and their uses.

Transgenic organisms, including transgenic animals, have been known for a number of years. Although the term has occasionally been applied to any organism which contains foreign DNA, the term “transgenic organism” is used herein in its more usual sense to denote eukaryotic organisms (and in particular, animals or plants, and especially vertebrates e.g. mammals) and their progeny which contain heterologous chromosomal DNA in the germ line. The heterologous chromosomal DNA comprises a coding sequence which is hereinafter referred to as a “transgene”. Thus, every (or at least most) of the cells of a transgenic organism—both somatic and germ—may contain one or more copies of the transgene(s).

Transgenic organisms can be produced by many different methods. The methods are well documented in the prior art and their practice forms part of the technical repertoire of those skilled in the art. Methodological approaches commonly used are described for example in First and Haseltine (Eds.), Transgenic Animals (1991) Butterworth-Heineman M A USA.

According to one known method, the transgene is inserted into embryonic stem cells which are then injected into fertilized zygotes at a stage when only a small number of cells are present. The engineered embryonic stem cells become incorporated into the zygote, and cells derived therefrom go on to differentiate into many or all of the different cell types of the animal's body. Such cells may also include those contributing to the germline, and the progeny of such (chimaeric) animals may therefore be fully transgenic.

Other methods involve the introduction of the transgene into the pronucleus or into the fertilized or unfertilized ovum.

It is also known in the art that cells can be routinely engineered or induced to express gene(s) which confer any of a wide variety of selectable phenotypes thereon. Such genes are known as selectable markers. They are normally introduced into cells as part of a recombinant expression vector. The selectable phenotype conferred by a selectable marker may be classed as either positive or negative.

A positive selectable phenotype is one which permits survival under particular conditions which would kill (or at least prevent or impair the growth of) cells which do not exhibit the positive selectable phenotype.

A negative selectable phenotype is one which results in the destruction (or the prevention or impairment of growth) of the cell under particular conditions which are relatively innocuous to cells which do not exhibit the negative selectable phenotype.

A wide variety of selectable markers are available. Genes that are widely applied as positive selectable markers include the bacterial neomycin phosphotransferase (neo; Colbere-Garapin et al. (1981) 150:1), hygromycin phosphotransferase (hph; Santerre et al. (1984), Gene 30:147) and xanthineguanine phosphoribosyl transferase (gpt; Mulligan and Berg (1981), P.N.A.S., USA 78:2072).

Also used as positive selectable markers are the Herpes simplex virus type 1 thymidine kinase (HSV-1 TK; Wigler et al. (1977), Cell 11:223), adenine phosphoribosyltransferase (APRT; Wigler et al. (1979), P.N.A.S. USA 76:1373) and hypoxanthine phosphoribosyltransferase (HPRT; Jolly et al. (1983), P.N.A.S. USA 80:477). These latter markers must be used in cells having a particular mutant genotype (viz. one which leads to a deficiency in the gene product on which the selection is based).

Some of the aforementioned genes also confer negative as well as positive selectable phenotypes. They include the HSV-1 TK, APRT, HPRT and gpt genes. These genes encode enzymes which can catalyse the conversion of certain nucleoside or purine analogues to cytotoxic intermediates. For example, the nucleoside analog ganciclovir (GCV), is a good substrate for the HSV-1 thymidine kinase but a poor substrate for the natural thymidine kinase found in mammalian cells. Consequently, GCV can be used for efficient negative selection against cells expressing the HSV-1 TK gene (St. Clair et al. (1987), Antimicrob. Agents Chemother. 31:844). Xanthineguanine phosphoribosyl transferase can be used for both positive and negative selection when expressed in wild type cells (Besnard et al. (1987), Mol. Cell. Biol. 7:4139).

Selectable markers are usually used in both prokaryotic and eukaryotic genetic engineering to permit the recovery, from a mixed population, of cells which have undergone a relatively rare genetic change. For example, they can be used in physical association with another gene which encodes a product of interest (for example, a therapeutic protein) to select cells which have taken up that other gene along with the selectable marker. For example, the neo gene has been used to monitor genetically modified cells taken from patient samples after gene therapy has taken place.

It has also been proposed to use negative selectable markers as a safety device in gene therapy. Many gene therapies involve the removal of somatic cells from the patient, the introduction therein of a therapeutic gene (the expression of which repairs a biochemical lesion), followed by reintroduction of the cells back into the patient. Since the reintroduced genetically modified cells may ultimately prove deleterious to the health of the patient (for example, if they prove to be immunologically incompatible or become malignant), a negative selectable marker may be introduced along with the therapeutic gene to permit (if necessary) subsequent selective elimination of the genetically modified cells.

A number of vectors bearing positive or negative selectable markers have been made and are readily available to those skilled in the art (for review see Miller (1992), Nature 357:455-460). Others may be readily assembled using standard gene cloning techniques.

Transgenic organisms bearing a selectable marker as a transgene are known in the art. Generally, the selectable marker is introduced to permit the recovery of cells which have also taken up a gene of interest to which the selectable marker was linked. For example, many such transgenic organisms have been constructed in the course of work involving the introduction into the germline of genetic information which disrupts normal development (see WO/91/13150).

Transgenic organisms bearing a selectable marker have also been constructed in the course of the construction of animals bearing a specific genetic lesion. Here, the selectable marker is inserted (usually by homologous recombination) into a particular gene which is thereby insertionally inactivated. The selectable phenotype conferred by the selectable marker is then used as the basis for the identification and recovery of cells bearing an insertionally inactivated copy of the gene.

Examples of transgenic animals produced by such methods are described in Piedrahita et al. (1992), PNAS 89, pages 4471-4475.

In vitro tissue/cell culture methods are fundamental to pharmaceutical, clinical, agricultural, and industrial research. For example, tissue/cell culture methods are used in the pharmaceutical industry for the preparation of medicaments (for example, therapeutically active protein products) and in assays or screens for potential drugs.

However, these in vitro cell/tissue culture techniques are slow, laborious and expensive. One important problem is culture infection (usually arising from microbial contamination by bacteria, yeasts and/or fungi). This is usually countered by the use of various antibiotics which are added to the culture medium to eliminate or reduce the growth of contaminants.

However, the use of antibiotics does not completely eliminate the risk of infection, especially that arising from yeast and/or fungal contaminants (many of which are resistant to the commonly used antibiotics).

Another important problem arises from the need for cultures of a single tissue or cell type. Growth in vitro from single cells may be difficult (often requiring the use of feeder cells and/or mixtures of growth factors and other supplements) and homogenous in vitro populations cannot therefore be easily obtained.

There is therefore a need for a convenient source of cells/tissue of all types for primary culture or other purposes.

It has now been found that transgenic organisms bearing positive and/or negative selectable markers have previously unrecognized utility in cell culture techniques.

The present invention provides transgenic organisms which inter alia constitute a very convenient source of material for the isolation, identification, culture and analysis of cells from any tissue of the organism's body. Tissue dissected from the transgenic organisms of the invention can be particularly easily grown (even as homogenous populations of a particular cell/tissue type) in vitro and used in a wide variety of applications, including pharmaceutical assays, tissue transplant synthesis, drug delivery and protein production.

According to one aspect of the present invention there is provided a transgenic eukaryotic organism having cells containing heterologous DNA comprising a transgene encoding a positive selectable marker and a transgene encoding a negative selectable marker. But for the selectable phenotypes arising from the transgenes, the organism may be essentially normal, the transgenes for example not being located such that they insertionally inactivate a gene.

The term “essentially normal” as used herein may indicate that the organism is not mutant for any significant character or trait with respect to the wild type and/or exhibits normal tissue differentiation and development. For example, the organism may be essentially normal in the sense that the transgenes are resident in a silent (i.e. non-expressed) region of the genome and/or in a region of the genome where the transgenes do not significantly perturb the replication, segregation, organisation or packing of the chromosome or its interaction with cellular components such as DNA binding proteins (including histones and regulatory elements).

The provision of transgenes encoding both positive and negative selectable markers provides great flexibility during subsequent manipulation of cells derived from the transgenic organism in vitro. Moreover, where the invention is used to generate tissue transplants, cells of a particular type may be isolated from the transgenic animal of the invention by positive selection. The cells so isolated may then be transplanted into a non-transgenic animal to determine whether the transplant has any therapeutic effect. The transplant may then be ablated by negative selection to provide a control to determine whether the transplant was having a direct therapeutic effect.

In another aspect, the invention provides a transgenic eukaryotic organism having cells containing heterologous DNA comprising a transgene encoding a positive selectable marker and/or a transgene encoding a negative selectable marker, the organism being essentially normal but for the selectable phenotypes arising from the transgene(s). A positive and negative selectable marker may be provided by a single transgene, since (as explained above) some markers can be used as both positive and negative selectable markers (depending upon the selection conditions used).

The transgenic eukaryotic organism of the invention is preferably an animal or a plant, for example a vertebrate (e.g. a mammal, for example a rat, rabbit, pig or mouse).

The transgenic organism preferably has a genotype which is essentially wild type but for the presence of the heterologous DNA.

In addition, that portion of the heterologous DNA which is expressed in the cells may consist of a transgene encoding a positive selectable marker and/or a transgene encoding a negative selectable marker, each transgene being operably linked to an expression element or elements. The absence of expression of any other transgenically derived genetic sequences makes this preferred transgenic organism suitable for a wide range of experimental research requiring an effectively wild type genetic background.

At least one of the selectable markers may be operably linked to a regulatable expression element or elements, for example a tissue- or cell-specific expression element or elements. In such circumstances, each selectable marker is advantageously differentially regulated, each marker for example being linked to a different tissue- or cell-specific expression element or elements. This permits the expression of the selectable marker to be limited to a selected class of cells or tissue, so providing e.g. for the selective culture in vitro of the selected class of cells or tissue from a mixed primary cell culture.

The present invention does not rely on the use of transgenic organisms produced by any one method: any transgenic procedure may be used in the practice of the invention. Moreover, in most circumstances, the precise nature of the selectable markers for use in the present invention is unimportant: in general, any selectable marker gene may be used so long as it confers a positive or negative selectable phenotype on the cell.

For example, the positive selectable marker may be selected from neomycin phosphotransferase, hygromycin phosphotransferase, xanthineguanine phosphoribosyl transferase, the Herpes simplex virus type 1 thymidine kinase, adenine phosphoribosyltransferase and hypoxanthine phosphoribosyltransferase.

The negative selectable marker may for example be selected from Herpes simplex virus type 1 thymidine kinase, adenine phosphoribosyltransferase, hygromycin phosphotransferase and hypoxanthine phosphoribosyltransferase.

The selectable markers are conveniently derived (e.g. by subcloning using restriction endonucleases) from any of a large number of known vectors, examples of which are described in e.g. Molecular Cloning: A laboratory Manual Second Edition Edited by Sambrook J, Fritsch and Maniatis T 1989 Cold Spring Harbour Laboratory Press).

The expression elements for use in the invention may take any form so long as they can (under at least some circumstances) be made to direct and/or control the expression of the genes with which they are operably coupled. Expression elements for use in the invention may comprise transcriptional and/or translational elements, and include promoters, ribosome binding sites, enhancers and regulatory sites including activator and repressor (operator) sites. Preferred expression elements comprise promoters selected from a wide range available for use, examples of which are shown in Table 1. This Table, which is non-exhaustive, also indicates the use to which each promoter may be put in the methods of the invention described infra.

By way of example only, the expression elements for use in the invention may be selected from: promoters and/or enhancers which are specifically active in: (i) dopaminergic, serotoninergic, GABAergic, cholinergic or peptidergic neurones and sub-populations thereof; (ii) oligodendrocytes, astrocytes and sub-populations thereof; (iii) the endocrine glands, lungs, muscles, gonads, intestines, skeletal tissue or part or parts thereof; (iv) epithelial, fibroblast, fat, mast, mesenchymal or parenchymal cells; (v) particular stages of embryogenesis, and (vi) components of the blood system (e.g. T-lymphocytes, B-lymphocytes and macrophages). Alternatively they may be selected from promoters and/or enhancers which direct the transcription of genes for: (i) neurotransmitter-specific receptors; (ii) ion channels; (iii) receptors involved in ion channel gating and (iv) cytokines, growth factors and hormones.

At least one of the selectable markers may advantageously be constitutively expressed. This ensures uniform expression of the selectable marker in every transgenic cell of the transgenic organism under all conditions, which is particularly useful where the transgenic organism is for general use as a source organism for cell/tissue culture.

TABLE 1

| Promoter | Tissue/cell-type | Application | Reference |
|---|---|---|---|
| Tyrosine | Catecholamin- | Alzheimer's | 1 |
| hydroxylase | ergic neurones | Parkinson's | |
| TSH | Thyroid cells | Hypothyroidism | 2 |
| receptor | | | |
| BSF1 | GABAergic | Epilepsy | 3 |
| | neurones | | |
| Human dopamine | Noradrenalin | Alzheimer's | 4 |
| 3-hydroxylase | neurones | | |
| Thyroglobulin | Thyroid cells | Hypothyroidism | 5 |
| Serotonin 2 | Glial cells in | Neuro- | 6 |
| receptor | serotoninergic | degenerative | |
| | projection areas | diseases | |
| Mouse inter- | bone cells and | inflammatory | 7 |
| leukin 4 | heaematopoietic | processes | |
| | system | | |
| CD4 receptor | CD4 expressing | AIDS | 8 |
| | T-lymphocytes | | |
| human choline | Acetylcholine | Alzheimer's | 9 |
| acetyltrans- | neurones | Motor-neurone | |
| ferase | | disease | |

References
1: Stachowick et al. (1994), Molecular Brain Research 22(1-4): 309-319.
2: Ikuyama and Nawata (1994), Japanese Journal of Clinical Medecine 52(4): 962-968
3: Motejlek et al. (1994), Journal of Biological Chemistry 269(21): 15265-15273
4: Hoyle et al. (1994), Journal of Neuroscience 14(5 Pt 1): 2455-2463
5: Pichon et al. (1994), Biochemical Journal, 298 (pt 3): 537-541
6: Ding et al. (1993), Molecular Brain Research, 20(3): 181-191
7: Bruhn et al. (1993), PNAS USA, 90(20): 90(20): 9707-9711
8: Nakayama et al. (1993), International Immunology, 5(9): 817-824
9: Li et al. (1993), Neurochemical Research, 18(3): 271-275
10: Sohanberg et al. (1991), PNAS, 99(2): 603-607

Constitutive expression may be achieved for example via the use of a promoter which directs the expression of a “house-keeping” gene. “A house-keeping” gene is one which is expressed in all cell types. Their translated products are required as part of general cell metabolism or cell structure and, consequently, they are not specifically expressed in a particular cell or tissue type. House-keeping gene promoters, therefore, need to be active in a broad range of (and sometimes in all) cell types in order to ensure constitutive gene expression. An example of a constitutively-expressed promoter useful in the present invention is that for the histocompatability complex H-2K^{b} class 1 promoter (Weiss et al. (1983) Nature, 301, 671-674; Baldwin and Sharp (1987), Mol. Cell. Biol. 7, 305-313; Kimura et al. (1986), Cell 44, 261-272) which has been shown to express downstream coding sequences in cells generally when used as a promoter in a transgene (Jat et al (1991), PNAS USA 88, 5096-5100). Another example is the viral SV40 early promoter.

The promoters for use in the present invention are not restricted to those derived from mammalian cells but may also include avian- and fish-derived promoters. Additionally, virally derived promoters, some of which have biological activity in a broad range of mammalian, fish and avian cells as well as other eukaryotes, could also be used in performing the invention. Examples are the simian virus-40 derived early or late promoters, or the Long Terminal Repeats (LTR'S) of retroviruses which comprise promoter as well as enhancer elements and have the ability to promote expression of sequences under their influence in a broad range of eukaryote cells. These promoters along with supporting sequences such as enhancer elements and other regulatory elements are well known to the man skilled in the art (see e.g. Molecular Cloning: A laboratory Manual Second Edition Edited by Sambrook J, Fritsch and Maniatis T 1989 Cold Spring Harbour Laboratory Press).

The transgenic organism of the invention may also contain heterologous DNA which further comprises a reporter transgene, for example 3-galactosidase or luciferase. The reporter transgene may be itself operably linked to an expression element or elements which are subject to cell- or tissue-specific regulation.

Such reporter transgenes facilitate subsequent analysis of cells/tissue cultured from the transgenic organism and in particular permit the response (to for example an induced deficit in a particular class of cells/tissue) of a particular expression element or class of expression elements to be monitored in vivo or in vitro.

In another aspect, the invention provides a method of culturing cells and/or tissues in vitro, comprising the steps of: (a) providing a transgenic animal or plant having cells containing genetic material which confers a selectable phenotype thereon; (b) generating a primary culture from cells and/or tissue of the transgenic organism of step (a); and (c) selectively growing the primary culture on the basis of the selectable phenotype conferred by the genetic material contained in the cells of the transgenic organism.

Preferably, the cell/tissue culture method of the invention is based on the use of a transgenic organism having a selectable marker operably linked to a tissue- or cell-specific expression element or elements, whereby in step (c) a particular cell/tissue type is selectively grown on the basis of the tissue- or cell-specific expression therein of said at least one selectable marker.

This preferred method of the invention finds application for example in the selection of thyroid follicular cells from a primary (mixed cell) culture. This method may provide a primary stromal cell population of the thyroid gland in the absence of the thyroid follicular cells and constitutes a unique cell culture system useful for the study of thyroid biology and in the development of new therapeutic drugs for the treatment of thyroid diseases.

The cell/tissue culture method of the invention may also be practised such that step (c) reduces or eliminates microbial contamination of the tissue culture, thereby alleviating or eliminating a common problem in cell culture systems, viz. culture contamination (particularly by fungi and yeasts).

The transgenic organisms of the invention can also be used as a source of lymphocytes in methods for the production of monoclonal antibodies.

Monoclonal antibodies are of fundamental importance in biotechnology. Their preparation involves a sequence of steps including: (a) immunizing an animal by injecting the antigen of interest, (b) removing the spleen from the animal and preparing lymphocytes therefrom, (c) fusing the lymphocytes with immortal (usually myeloma) cells to produce a hybridoma, (d) selectively growing hybridomas and (e) cloning the hybridomas to produce a clone secreting the monoclonal antibody of interest.

The step of selectively growing the hybridoma (step (d), above) is usually achieved on the basis of a HPRT⁻ genotype in a myeloma fusion partner which prevents unfused myeloma cells from growing in selective media containing hypoxanthine, aminopeterin and thymidine (HAT medium). This restricts the choice of fusion partners.

Thus, according to a further aspect of the present invention, there is provided a method of making a monoclonal antibody specific for an antigen, comprising the steps of: (a) providing a transgenic animal (for example a rat, rabbit, pig or mouse) having lymphocytes containing genetic material which confers a selectable phenotype thereon; (b) immunizing the transgenic organism with the antigen; (c) removing the lymphocytes from the transgenic animal; (d) fusing the lymphocytes of step (c) with immortal cells (for example tumour cells, e.g. myeloma cells) to produce hybridomas; and (e) selectively culturing the hybridomas on the basis of the selectable phenotype conferred by the genetic material contained in the lymphocytes.

The presence of the selectable marker in the lymphocyte preparations from the transgenic animal obviates the requirement for eg. a HPRT selection process and expands the repertoire of fusion partner cells that can be used in hybridoma formation.

The transgenic organisms of the present invention also find application in relation to diseases or disorders involving cell loss.

Many diseases and disorders are known to be associated with specific cell and/or tissue loss. For example, in neurodegenerative disorders such as Parkinson's disease, Huntington's chorea and Alzheimer's disease one or more sub-populations of neurotransmitter-identified cells are lost during the course of the disease.

In Parkinson's disease, this loss is principally of the dopaminergic neurones of the substantia nigra region of the brain, although other cell types also decline.

In Huntington's chorea, there is a more general loss of neurones, but in this case the deficits are restricted largely to the striatum.

In Alzheimer's disease, there is a decrement in acetylcholine-, serotonin- and noradrenaline-containing neurones projecting to the neo- and palaeocortex.

Other neurological diseases and disorders also stem from neural cell degeneration; the demyelination occurring in multiple sclerosis, for instance, is due to the destruction of oligodendrocytes in the brain.

The Human Immunodeficiency Virus (HIV) is known to enter cells that express the CD4 receptor and cell infection appears to lead ultimately to cell death. The loss of CD4 cells causes a catastrophic block of the entire immune system and death of the infected person.

The molecular/cellular basis of HIV induced-disease is poorly understood. This is due, at least in part, to the lack of model systems to study the pathogenesis of the disease, particularly in-vivo.

The use of SIV (simian immunodeficiency virus) infected primates has been considered as a paradigm, but SIV monkeys do not acquire full-blown AIDS. In many instances, they show no symptoms at all. Alternative models that have been proposed include HIV-infected chimpanzees. Apart from the potential ethical considerations, the manisfestation of AIDS-like symptoms in such a model may take several years, substantially hindering research and the development of effective therapies.

Thus, animal models of the various diseases and disorders discussed above are essential as test subjects for potential pharmaceuticals and in basic clinical research. The choice of these animal models is presently very limited because of the difficulties associated with selectively destroying specific cell and/or tissue types.

Thus, according to a further aspect of the present invention there is provided a method of selectively eliminating or depleting a particular tissue or cell type in an organism, comprising the steps of: (a) providing a transgenic organism having a negative selectable marker operably linked to an expression element (e.g. a promoter) specific for the tissue or cell type to be eliminated or depleted, and (b) administering a selective agent to the organism to eliminate or deplete that tissue or cell type on the basis of the expression therein of the negative selectable marker.

The selective agent may be administered by any route. Where systemic administration is required, oral, parenteral or intravenous routes may be used. Where localized administration is required (for example where the tissue or cell-type to be eliminated is restricted to a particular organ or to a particular region of the body) targeted injection, implantation (e.g. slow release capsules) or catheterization may be used. For example, tissue in particular regions of the brain may be specifically targeted by intracerebral injection.

The method of selectively eliminating or depleting a particular tissue or cell type of the invention may be employed to provide in vivo models of diseases/disorders involving disease-related cell loss, for example immunodegenerative or neurodegenerative diseases/disorders (such as AIDS, Parkinson's and Alzheimer's disease).

Accordingly, in a further aspect the present invention provides a method of modelling disease/disorder-related cell/tissue loss or atrophy comprising the steps of: (a) providing a transgenic organism having a negative selectable marker operably linked to an expression element (e.g. a promoter) specific for the tissue or cell type which is subject to disease-related elimination or atrophy; and (b) administering a selective agent to the organism to eliminate or deplete the tissue or cell type on the basis of the expression therein of the negative selectable marker.

The invention also provides a method (e.g. an in vitro method) of determining the effect of a deficit in a first class of cells on the characteristics of a second class of cells in an organism, the method comprising the steps of: (a) providing a transgenic organism having a first negative selectable marker operably linked to an expression element specific for the first class of cells and either; (i) a positive selectable marker operably linked to an expression element specific for the second class of cells, or (ii) a second negative selectable marker linked to an expression element which directs the expression of the negative selectable marker in all cells of the organism except the second class of cells; (b) administering a selective agent to the organism to induce a deficit in the first class of cells on the basis of the expression therein of the negative selectable marker; (c) removing cells from the organism; and (d) selectively culturing cells of the second class from those cells removed in step (c) on the basis of; (i) the expression therein of the positive selectable marker, or (ii) the lack of expression therein of the negative selectable marker.

In another aspect the invention provides a method of screening compounds for pharmacological activity against a disease or disorder involving cell/tissue loss or atrophy, comprising the steps of: (a) providing a test model of the disease via the steps of; (i) providing a transgenic organism having a negative selectable marker operably linked to an expression element (e.g. a promoter) specific for the tissue or cell type which is subject to disease/disorder-related elimination or atrophy, and then (ii) administering a selective agent to the organism to eliminate or deplete the tissue or cell type on the basis of the expression therein of the negative selectable marker to produce a test model; (b) administering the compound to be tested to the test model; (c) screening the compound to be tested on the basis of its effect on the test model of step (a).

The methods of the invention may be usefully applied to any disease or disorder which is associated with cell/tissue loss or atrophy. In particular, the methods of the invention find particular utility in respect of neurodegenrative or immunodegenerative diseases and disorders, for example: (a) Parkinson's disease (the tissue or cell-type to be eliminated or depleted comprising dopaminergic neurones in the substantia nigra); (b) Huntington's chorea (the tissue or cell-type to be eliminated or depleted comprising neural cells of the striatum; (c) Alzheimer's disease (the tissue or cell-type to be eliminated or depleted comprising acetylcholine-, serotonin- and/or noradrenaline-neurones associated with the neo- and palaeocortex; (d) multiple sclerosis (the tissue or cell-type to be eliminated or depleted comprising brain oligodendrocytes), (e) immune disease and the cell-type to be eliminated or depleted comprises CD3, CD4 and/or CD8 cells and (f) AIDS and the cell-type to be eliminated or depleted comprises CD4 cells.

In the case of AIDS models, the method of the invention could be used to specifically deplete or eliminate CD4 cells by linking a negative selectable marker to a CD4 cell-specific promoter (e.g. the CD4 receptor promoter). This would permit the generation of an in vivo model of AIDS by regulating the proportion of cells expressing CD4 by negative selection in vivo.

Furthermore, in the case where the transgenic animal model carries both a positive and negative selectable marker, any residual CD4 expressing cells could later be isolated from the transgenic tissue of the animal model by positive selection in vitro for further study.

Examples of various promoters suitable for use in the methods of the invention described above are listed in Table 1, along with the disease(s) in which each promoter may find application.

The invention also contemplates cell/tissue cultures derived from the transgenic organisms of the invention (or produced by the cell culturing methods of the invention), and also to various therapeutic uses of the invention.

The invention will now be described in more detail by way of specific examples. These examples are not intended to be taken as limiting in any way.

The methods and technologies required to construct plasmid vectors, for example, in order to generate the invention are well known to the man skilled in the art. The constructed sequences given below represent examples of numerous constructs that could be used to perform the invention. The invention should not be construed as being limited to their use only.

A. Materials

i. Vectors

| | |
|---|---|
| pBabeneo plasmid vector | Morgenstern, J. P. & Land, H. |
| | Nucl. Acids Res. 18(1990) 3587- |
| | 3596 (plasmid is freely |
| | available). |
| pCI plasmid vector | Promega, 2800 Woods Hollow Rd, |
| | Madison, USA |
| CD2 plasmid vector | Blaese, M. R., NIH, Bethesda, USA |
| | (plasmid freely available). |
| | Mullen, C. A., Kilstrup, M., |
| | Blaese, R. M., Proc. Natl. Acad. |
| | Sci. USA., 89(1992) 33-37. |
| | Austin, E. A. & Huber, B. E. Mol. |
| | Pharmacol., 43(1993) 380-387. |
| | Wallace, P. M., MacMaster, J. F., |
| | Smith, V. F., Kerr, D. E., Senter, |
| | P. D. & Cosand, W. L. Cancer Res. |
| | 54(1994) 2719-2723. |
| TG-TKα plasmid vector | Wallace, H., Kings Buildings, |
| | University of Edinburgh, UK |
| | (plasmid freely available). |
| | Wallace, H., Ledent, C., |
| | Vassart, G., Bishop, J. O. & |
| | AlShawi, R. Endocrinology, |
| | 129(1991) 3217-3226. |
| pPBS plasmid | Morgan, Nucleic Acids Research |
| | (1992), 20, pages 1293-1299 |
| ii. Molecular Biology Reagents | |

| | |
|---|---|
| Restriction endo- | Promega, 28000 Woods |
| nucleases | Hollow Rd, Madison, USA |
| DNA modifying enzymes, | Promega, 28000 Woods Hollow |
| ligase, CIP, T4 | Rd, Madison, USA |
| polymerase etc. | |
| Agarose for electo- | Sigma Chemical Co., St. |
| phoresis | Louis, USA |
| Polynucelotide kinase | New England Biolabs Ltd., |
| and buffers | 3397 American Drive, Unit 12, |
| | Mississauga, Ontario, Canada |

B. Construction of Genes

(i) Thyroglobulin-Thymidine Kinase-Internal Ribosomal Entry Site-Neomycin Resistance (TG-TrK-α-IRES-neo^{r})

The neomycin resistance gene (neo^{r}) was obtained from the pBabe Neo plasmid (Morgenstern & Land, Nucl. Acids Res. 18(1990)3587-3596) by digestion with Hind III/Cla I and retrieval of the 1165 b.p. fragment containing the neo^{r} gene by gel electrophoresis and the Promega Wizard PCR kit.

The pPBS plasmid (Morgan, Nucl. Acids Res. (19902) 20, pages 1293-1299) comprising the poliovirus derived internal ribosomal entry site sequence was digested with Hind III/Cla I. However, this could not be done simultaneously, or, in sequence, since the restriction sites were too close together. In order to overcome this problem, the plasmid was initially digested with Hind III and a 200 b.p. fragment of DNA containing Hind III restriction sites at both the 5′ and 3′ ends was inserted in order to separate the sites. The pPBS plasmid could then be digested first with Cla I and then with Hind III.

Terminal phosphate groups were removed from the Hind III/Cla I cut pPBS vector using calf intestinal phosphatase (CIP). The vector was gel-purified using a 1% agarose gel and a band containing the DNA was excised and electroeluted.

The neomycin gene was then ligated into the pPBS plasmid overnight at 15° C. and the ligation reaction transformed into freshly-made MC1061 competent cells.

Positive colonies were identified by digestion of prepared plasmids with Hind III/Cla I. The neo^{r} gene and plasmid being detected electrophoretically in plasmid preparations from positive colonies. Plasmids from the positive colonies were then digested with Hinc II and Sac I (both restriction enzymes leaving digested DNA with blunt ends). The resulting Sac I/Hinc II digestion containing the IRES-neo^{R} fragment was run on a 1% electrophoresis gel and the appropriate size band was excised and the DNA electroeluted and ethanol-precipitated.

The TG-TKα plasmid (freely available from Genbank, NIH, USA accession No. JO2224, Santelli et al 1993) DNA was prepared using Promega Wizard mini preps and digested with Nar I. The ends of the plasmid were blunted using T4 Polymerase at 37° C. for 1 h followed by removal of the terminal phosphate groups using CIP. The CIP was inactivated by treatment of the DNA with phenol/chloroform followed by ethanol precipitation. The resulting plasmid was electrophoresed on a 1% agarose gel and the DNA was recovered and ligated with the insert in a 1:3 molar ratio of plasmid to insert.

The ligation was incubated at 15° C. overnight, and was then used to transform competent MC1061 cells. Positive colonies were selected by digestion of prepared plasmids with BamH I (the correct construct provided restriction fragments of size 3980, 1663, 3102 and 1039 b.p.).

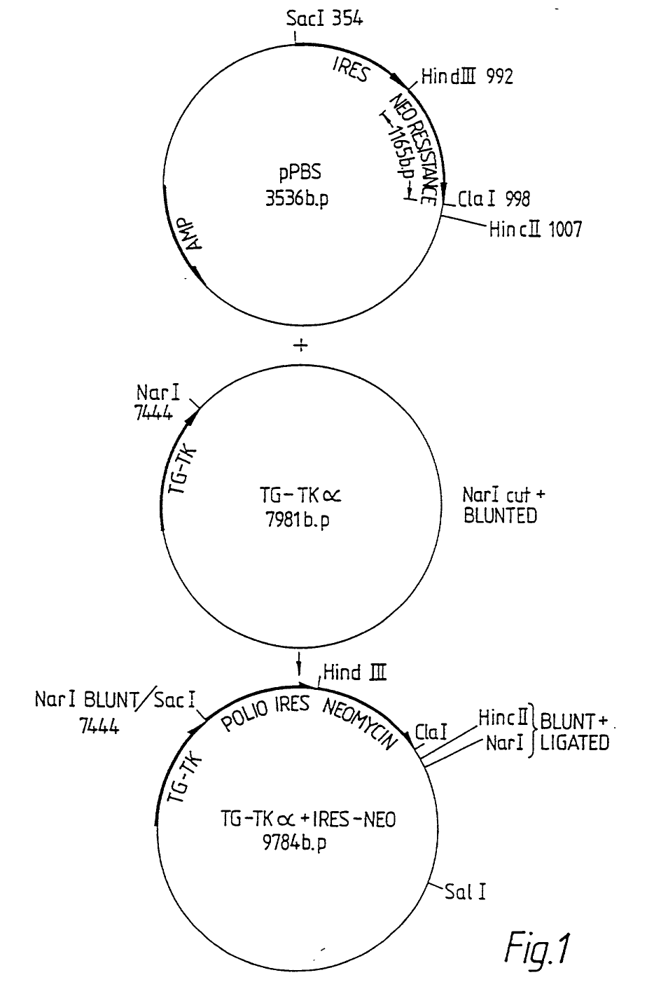

Linearization of the plasmid was achieved by digestion of prepared plasmids with Sal I restriction enzyme. The construction is shown in FIG. 1.

(ii) Cytomegalovirus-Cytosine Deaminase-sv40 Promoter-Neomycin Resistance (CMV-CD-SV40-neo^{r}, or CD2-neo^{r})

pCD2 plasmid (Mullen et al., PNAS 89(1992)33-37) was digested with EcoR I and EcoR V, and the digest was electrophoresed on a 1% agarose gel where the 2.5 kb. Fragment containing the cytosine deaminase gene, the SV40 promoter and the neomycin resistance gene was retrieved by electroelution followed by ethanol preciptation.

To ensure terminal phosphate groups were present in the fragment it was treated with polynucleotide kinase.

The pCI vector was digested with EcoR l and Sma I (a restriction enzyme leaving the DNA with blunt ends), and the terminal phosphate groups were removed using CIP and the enzyme was inactivated with phenol/chloroform followed by ethanol precipitation. The band was then gel-purified and recovered by electroelution.

The ligation was set up containing a 3:1 molar ratio of insert to vector and was carried out at 15° C. overnight. The ligation mixture was used to transform freshly-prepared MC1061 competent cells and positive colonies were selected by digestion of prepared plasmids with EcoR l and Hind III to provide restriction fragments of length 1868 b.p. and 5062 b.p., respectively. Linearization of the plasmid was achieved by digestion with Bgl I.

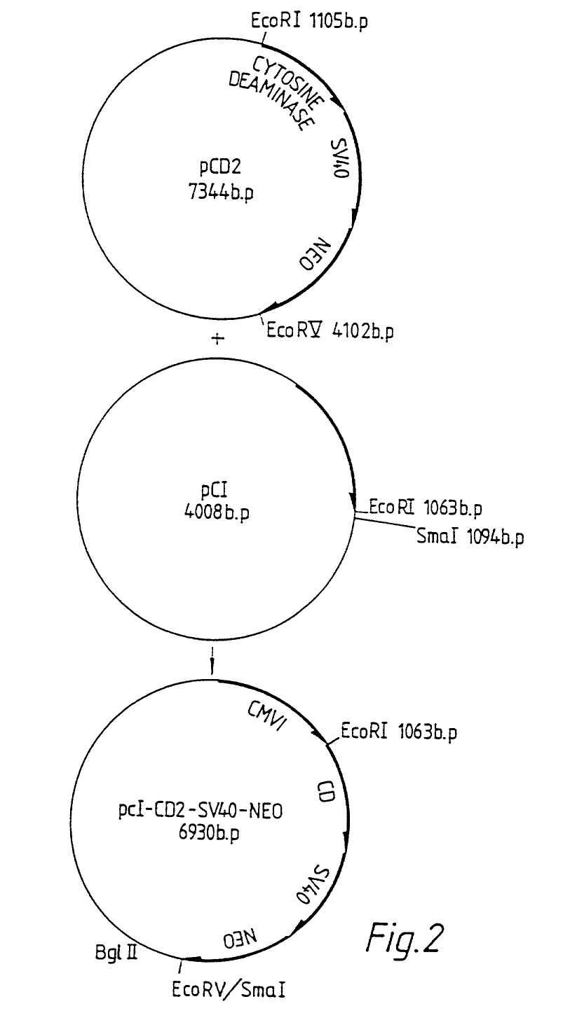

The construction is shown in FIG. 2.

C. Production of Transgenic Animals

Transgenic rats were produced by established methods (Hogan, B., Constantini, F. & Lacy, E. (1986) Manipulating The Mouse Embryo—A Laboratory Manual, Cold Spring Harbor Lab., Cold spring Harbor, N.Y.). In brief, approximately 2 pl of the plasmid were microinjected at a concentration of 5 μg/ml into the pronucleus of outbred Sprague-Dawley embryos. Embryos were then implanted into pseudopregnant recipients, and after identification of transgenic animals, lines were isolated and established. Lines were maintained as transgenic hemizygotes by mating hemizygous females with non-transgenic males.

D. Positive/Negative Selection of Cells from Transgenic Animals In Vitro

i. Fibroblast Cells

Fibroblast cultures derived from lung of adult CD2/neo^{r}, TG/TK/neo^{r} and control animals were produced and expanded by routine methods (Freshney (1987), Alan R. Liss, New York). Twenty-four hours after plating, geneticin (400 μg/ml) was added to cultures originating from both types of transgenic rats and from control rats, and replaced every three days with fresh medium. When required, cells were subcultured (1:3) to prevent them becoming confluent, again by basic culture methods (Freshney 1987). Cell counts were made manually in 20 fields chosen randomly and the values at each time point, after allowing for changes due to subculturing, were aggregated. As can be seen from Table 2, no fibroblast cells derived from control animals or the TG/TK/neo^{r} transgenic survived more than 10 days treatment with geneticin. In the absence of added geneticin, no change in cell survival from either of the transgenic animals was observed.

The effects of 5-fluorocytosine (5FC) were also determined. 5-fluorocytosine at a concentration of 100 μg/ml had no effect on fibroblast cells derived from control animals or from the TG/TK/neo^{r} transgenic. In the cells derived from the CD2/neo^{r} transgenic animal, however, 94% of the originally-plated cells died, or were non-functional (as determined by their failure to exclude trypan blue) after 10 days culture in the presence of 5FC (Table 2). By contrast, no significant difference in cell counts was found between cultures from control rats in the absence and presence of 5FC, or between controls and cultures taken from CD2/near rats in the absence of added 5FC (Table 2).

TABLE 2
Survival of lung fibroblast cells derived from
control and transgenic rats, and effects of
various drugs.

| | Days in culture | | | | | |
|---|---|---|---|---|---|---|
| Genotype/drug | 1 | 3 | 5 | 7 | 9 | 11 |
| Control | 100 | 100 | 100 | 100 | 100 | 100 |
| TG/TK/neo⁼ | 98 | 97 | 98 | 95 | 95 | 96 |
| CD2/neo⁼ | 92 | 93 | 92 | 98 | 105 | 98 |
| Control + geneticin | 99 | 101 | 85 | 23 | 5 | 2 |
| TG/TK/neo⁼ + geneticin | 97 | 105 | 108 | 101 | 105 | 111 |
| CD2/neo⁼ + geneticin | 91 | 94 | 91 | 93 | 107 | 105 |
| Control = 5FC | 101 | 105 | 98 | 97 | 93 | 96 |
| TG/TK/neo⁼ +5FC | 98 | 96 | 95 | 92 | 92 | 93 |
| CD2/neo⁼ + 5FC | 94 | 5 | 3 | 3 | 4 | 4 |

Drugs were added at day 2 in culture. Values are related to the number of cells found in control cultures without drug additions at various times after plating, and allowing for dilutions resulting from passaging. Figures are the means of three separate determinations, the standard errors all being less than 15% of the mean.

ii. Thyroid Cells

Thyroid cultures derived from the thyroid gland of adult CD2/neo^{r}, TG/TK/neo^{r} and control animals were produced by routine methods (Freshney, 1987). Twenty-four hours after plating, geneticin (400 μg/ml) was added to cultures originating from both types of transgenic and the control rats, and replaced every three days with fresh medium. When required, cells were subcultured (1:2) to prevent their becoming confluent. Cell counts were made manually in 20 fields chosen randomly, and the values at each time point, after allowing for changes due to subculturing, were aggregated (Table 3). Ten days after the initial application of geneticin, 10 μg/ml acycloguanosine (ACG, Sigma) was added to thyroid cells originating in the TG/TK/neo^{r} transgenic. Ten days later, cell counts were again made of 20 fields chosen at random. Results are given in Table 3. To summaraize, cells derived from both types of transgenic animal survived the geneticin treatment, whereas the control cells did not. Cells derived from the TG/TK/neo^{r} transgenic did not survive ACG treatment, whereas the cells derived from the control animals did. The results were as expected in view of the specific and non-specific expression of the positive and negative selection markers, in the TG/TK/neo^{r} and CD2/neo^{r} transgenics, respectively. TG/TK/neo^{r} transgenic rat thyroid cells cultured in the absence of any added drug did not exhibit any differences in their growth or survival compared to control thyroid cell cultures (Table 3).

TABLE 3
Survival of thyroid cells derived from control
and transgenic rats, and effects of various
drugs.

| | Days in culture | | | | | |
|---|---|---|---|---|---|---|
| Genotype/drug | 1 | 3 | 5 | 7 | 9 | 11 |
| Control | 100 | 100 | 100 | 100 | 100 | 100 |
| TG/TK/neo⁼ | 91 | 95 | 93 | 92 | 101 | 99 |
| CD2/neo⁼ | 99 | 103 | 102 | 97 | 89 | 91 |
| Control + geneticin | 95 | 91 | 85 | 56 | 9 | 4 |
| TG/TK/neo⁼ + geneticin | 104 | 105 | 98 | 88 | 93 | 98 |
| CD2/neo⁼ + geneticin | 91 | 94 | 91 | 93 | 107 | 105 |
| Control = ACG | 94 | 97 | 98 | 91 | 92 | 102 |
| TG/TK/neo⁼ + ACG | 98 | 38 | 12 | 10 | 8 | 7 |
| CD2/neo⁼ + ACG | 98 | 90 | 93 | 93 | 97 | 88 |

Drugs were added at day 2 in culture. Values are related to the number of cells found in control cultures without drug additions at various times after plating, and allow for dilutions resulting from passaging. Figures are the means of three separate determinations, the standard errors all being less than 15% of the mean.

E. Enhanced Sterility in Tissue Culture using Cells from CD2-neo^{r} Transgenic Animals

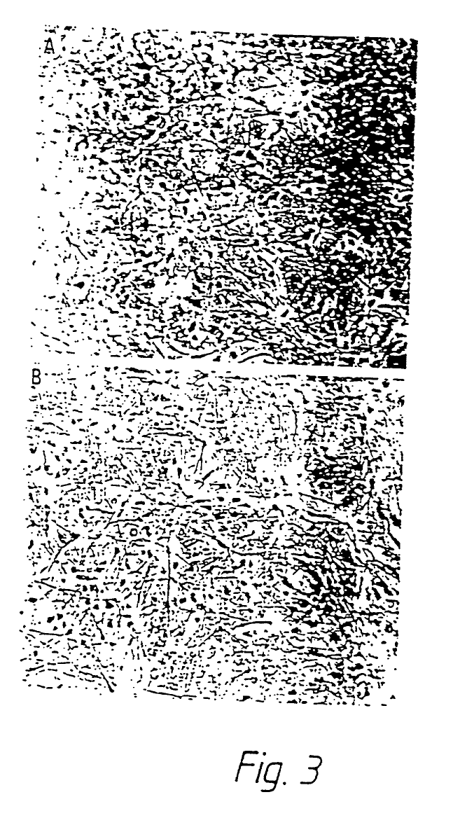

Cultures of spinal cord cells derived from the CD2-neo^{r} transgenic rat were grown in small flasks, using previously established methods (Foster et al., (1990) Eur. J. Neurosci. 3, 32-39). At the beginning of the experimental period, in the region of 100 yeast spores, and unknown amounts of other common laboratory microbiological contaminants, were introduced into the flask. At the same time geneticin (1 mg/ml) was added. Thereafter, all manipulations of the medium and additions to the flask were conducted outside the sterile environment of the laminar flow cabinet. Sixty days after the initial plating, no evidence of any form of contamination was apparent. Indeed, the survival and development of the neural cells appeared unimpaired compared to uninfected control spinal cord cells grown in the absence of geneticin (see FIG. 3, which shows phase-contrast photomicrographs of 60 days in vitro spinal cord cultures derived from control rat embryos (A) and from CD2/neo^{r} transgenic rat embryos (B) at day 14 of gestation. The latter cultures were deliberately infected with yeast and other microorganisms, and simultaneously treated with geneticin. No infection could be discovered after geneticin application, whereas infected cultures without geneticin were overrun with microorganism growth within 4 or 5 days).

F. Ablation of Thyroid Follicle Cells In Vivo

Adult female rats (250 g) were injected intra-peritoneally with 50 mg of ACG per day for a period of 5 days. Seven days after the final injection, serum levels of T₃ and T₄ were measured (Amersham, UK), and found to have fallen in transgenic animals from 0.76@0.05 nM to less than 0.06 M (T₃) and from 58.2@3.2 nM to less than 2.5 nM (T₄) (N=6). Administration of saline to transgenic animals resulted in a small but non-significant fall in T₄ to 0.68@0.07 nM (N=6). The thyroid glands of transgenic rats treated with ACG for 12 days had shrunk to 7% of the original weights. Histochemical analysis of these thyroid glands revealed an almost complete loss of follicular cells, with only non-follicular, perhaps calctonin-producing cells, remaining. Administration of lower amounts of ACG per day resulted in a partial loss of T₃ and T₄. In most other tissues from transgenic animals, HSV-thymidine kinase activity (Brinster et al. (1981) Cell 27, 223-231; Jamieson et al. (1974) J. Gen. Virol. 24, 481-492) was not expressed in detectable amounts. No histochemical evidence of cell loss was demonstrable in parathyroid, submaxillary or adrenal glands, nor in heart, kidney or brain.

In summary, both types of transgenic animal, or the cells therefrom, were apparently normal until application of either ACG or 5FC, as appropriate. After such application, either in vivo or in vitro, the cells upon which sensitivity had been conferred were rapidly destroyed. In addition, cells from both transgenic animals were resistant to the cytotoxic effects of geneticin, whereas cells from non-transgenic controls were completely eradicated.

### EXAMPLE 2

### Proposed Protocol for the Production of a Transgenic Mouse Bearing both Positive and Negative Selectable Markers

The herpes simplex virus (HSV) thymidine kinase gene (tk) (operably linked to the tk promoter) and the bacterial neomycin phosphotransferase (neo) gene (operably linked to the SV40 early promoter) are cloned into the appropriate cloning sites of a plasmid vector.

The plasmid vector is digested with restriction endonucleases and a fragment containing both the tk and neo selectable markers (along with the expression elements operably linked thereto) is isolated on an agarose gel.

The fragment isolated on the gel is then purified and injected into male pronuclei of fertilized one-cell mouse eggs at a concentration of 1-2 ug/ml DNA in TE buffer (10 mM Tris, Ph 7.5, 0.2 Mm EDTA). The eggs are those derived from a CBA×C57BL/10 mating.

The eggs which survive micro-injection are then transferred to pseudopregnant females as described e.g. in Wagner et al. (1981) PNAS 78, 5016, and allowed to develop to term.

At 7-14 days of age, each pup is analysed to determine whether the transgenes are present. DNA is prepared from a section of the tail by the method described in Sambrook et al. (1989) “Molecular Cloning”, Cold Spring Harbor. The presence of the neo and tk genes is determined by probing with labelled tk and neo-specific probes.

The transgenic pups so identified are mated and their offspring also analysed to check for Mendelian transfer of the transgenes.

### EXAMPLE 3

### Proposed Protocol for the Selective Culture of Mouse Thyroid Follicular Cells

Transgenic mice are prepared as described in Example 1, except that the neo gene is placed under the control of a thyroglobulin promoter (e.g. described by Christophe et al. (1989) Molecular and cellular endocrinology 64(1) 5-18; Christophe et al (1987), 19, Suppl. 17, pp 70-73; and Ledent et al. (1990), PNAS, 87 (16), pp 6176-6180).

The transgenic mice are sacrificed and the thyroid tissue removed and a primary culture Prepared in the presence of antibiotic G418. This antibiotic kills cells not expressing the neo gene, and results in the selective culturing within the primary (mixed cell) culture of thyroid follicular cells.

### EXAMPLE 4

### Proposed Protocol for the Preparation of Monoclonal Antibody

The bacterial neomycin phosphotransferase (neo) gene (operably linked to the SV40 early promoter) is cloned into the appropriate cloning site of a plasmid vector.

The plasmid is then digested with restriction endonucleases and a fragment containing the neo selectable marker is isolated on an agarose gel, and transgenic mice bearing the neo transgene are then prepared essentially as described in Example 1.

The antigen against which a monoclonal antibody is required is purified and injected into the transgenic mouse prepared as described above along with Freund's adjuvant. The mouse is then sacrificed and the spleen removed and placed in tissue culture fluid. The spleen is teased apart to release the lymphocytes and these are isolated by centrifugation. The lymphocytes are then mixed with a myeloma fusion partner in the presence of polyethylene glycol to induce fusion and produce hybridomas.

Hybridomas are selected by supplementing the culture medium with the antibiotic G418 on the basis of the presence of the neo selectable marker in the mouse lymphocytes. The hybridomas are then cloned by limiting dilution and the relevant clone identified by screening via the appropriate binding assay.

The myeloma cell line does not need to have a negative selectable marker (e.g. HPRT⁻) Moreover, the presence of G418 in the culture medium reduces or eliminates the risk of culture infection.

### EXAMPLE 5

### Proposed Protocol for the Preparation of a Rattine Model of Parkinson's Disease

The herpes simplex virus (HSV) thymidine kinase gene (tk) is operably linked to a promoter which is active only in dopaminergic neurones in the substantia nigra and cloned into the appropriate cloning site of a plasmid vector.

The plasmid is digested with a restriction endonuclease and a fragment containing the tk selectable marker is isolated on an agarose gel, and transgenic rats bearing the tk transgene are then prepared essentially as described in Example 1.

Ganciclovir is then administered by injection into the substantia nigra regions of the brain of the transgenic rats to specifically eliminate or deplete the dopaminergic neurones expressing the negative selectable tk marker, thus providing a rattine model of Parkinson's disease.

### EXAMPLE 6

### Proposed Protocol for the Preparation of a Rattine Model of Alzheimer's Disease

The herpes simplex virus (HSV) thymidine kinase gene (tk) is operably linked to a promoter which is active only in acetylcholine-, serotonin- and/or noradrenaline-neurones associated with the neo- and palaeocortex is cloned into the appropriate cloning site of a plasmid vector.

The plasmid is digested with a restriction endonuclease and a fragment containing the tk selectable marker is isolated on an agarose gel, and transgenic rats bearing the tk transgene are then prepared essentially as described in Example 1.

Ganciclovir is then administered by injection into the appropriate region of the brains of the transgenic rats to specifically eliminate or deplete the acetylcholine-, serotonin- and/or noradrenaline-neurones associated with the neo- and palaeocortex expressing the negative selectable tk marker, thus providing a rattine model of Alzheimer's disease.

1. A transgenic eukaryotic organism having cells containing heterologous DNA comprising a transgene encoding a positive selectable marker and a transgene encoding a negative selectable marker, the organism being e.g. essentially normal but for the selectable phenotypes arising from the transgenes.

2. A transgenic eukaryotic organism having cells containing heterologous DNA comprising a transgene encoding a positive selectable marker and/or a transgene encoding a negative selectable marker, the organism being essentially normal but for the selectable phenotypes arising from the transgene(s).

3. A transgenic organism according to claim 1 or claim 2 which is an animal or a plant, for example a vertebrate (e.g. a mammal, for example a rat, rabbit, pig or mouse).

4. A transgenic organism according to any one of the preceding claims having a genotype which is essentially wild type but for the presence of the heterologous DNA and/or wherein that portion of the heterologous DNA which is expressed in the cells consists of a transgene encoding a positive selectable marker and/or a transgene encoding a negative selectable marker, each transgene being operably linked to an expression element or elements.

5. A transgenic organism according to any one of the preceding claims wherein at least one of the selectable markers is operably linked to a regulatable expression element or elements, for example a tissue- or cell-specific expression element or elements.

6. A transgenic organism according to claim 5 wherein each selectable marker is differentially regulated, each marker for example being linked to a different tissue- or cell-specific expression element or elements.

7. A transgenic organism according to any one of the preceding claims wherein at least one selectable marker is constitutively expressed.

8. A transgenic organism according to any one of the preceding claims wherein the heterologous DNA further comprises a reporter transgene, for example 3-galactosidase or luciferase.

9. A transgenic organism according to claim 8 wherein the reporter transgene is operably linked to an expression element or elements which are subject to cell- or tissue-specific regulation.

10. A transgenic organism according to any one of the preceding claims wherein:
(a) the positive selectable marker is selected from neomycin phosphotransferase, hygromycin phosphotransferase, xanthineguanine phosphoribosyl transferase, the Herpes simplex virus type 1 thymidine kinase, adenine phosphoribosyltransferase and hypoxanthine phosphoribosyltransferase and/or the negative selectable marker is selected from Herpes simplex virus type 1 thymidine kinase, adenine phosphoribosyltransferase, hygromycin phosphotransferase and hypoxanthine phosphoribosyltransferase, and/or
(b) the expression element is selected from:
(I) promoters and/or enhancers which are specifically active in: (i) dopaminergic, serotoninergic, GABAergic, cholinergic or peptidergic neurones and sub-populations thereof; (ii) oligodendrocytes, astrocytes and sub-populations thereof; (iii) the endocrine glands, lungs, muscles, gonads, intestines, skeletal tissue or part or parts thereof; (iv) epithelial, fibroblast, fat, mast, mesenchymal or parenchymal cells; (v) particular stages of embryogenesis, and (vi) components of the blood system (e.g. T-lymphocytes, B-lymphocytes and macrophages); or
(II) promoters and/or enhancers which direct the transcription of genes for: (i) neurotransmitter-specific receptors; (ii) ion channels; (iii) receptors involved in ion channel gating and (iv) cytokines, growth factors and hormones.

11. Tissue or cells derived or cultured from the transgenic organism of any one of the preceding claims.

12. A method of culturing cells and/or tissues in vitro, comprising the steps of:
(a) providing a transgenic animal or plant having cells containing genetic material comprising a selectable marker which confers a selectable phenotype on the cells, for example a transgenic animal or plant according to any one of claims 1 to 10;
(b) generating a primary culture from cells and/or tissue of the transgenic animal or plant of step (a); and
(c) selectively growing the primary culture on the basis of the selectable phenotype conferred by the genetic material contained in the cells of the transgenic animal or plant.

13. A method according to claim 12 wherein at least one selectable marker is operably linked to a tissue- or cell-specific expression element or elements, whereby in step (c) a particular cell/tissue type is selectively grown an the basis of the tissue- or cell-specific expression therein of said at least one selectable marker, e.g. to produce a homogeneous population of a particular class of cells in primary culture.

14. A method according to claim 12 or claim 13 whereby step (c) reduces or eliminates microbial (e.g. yeast and fungal) contamination of the tissue culture.

15. Tissue or cells cultured by the method of any one of claims 12 to 14, the tissue or cells being for use e.g. as a tissue transplant, as a test subject in biochemical assays or as a source of a protein of interest.

16. Tissue or cells according to claim 11 or claim 15 for use in therapy.

17. A method of making a monoclonal antibody specific for an antigen, comprising the steps of:
(a) providing a transgenic animal (e.g. a transgenic animal according to any one of claims 1 to 10) having lymphocytes which contain genetic material which confers a selectable phenotype thereon;
(b) immunizing the transgenic organism with the antigen;
(c) removing the lymphocytes from the transgenic animal;
(d) fusing the lymphocytes of step (c) with immortal cells (for example tumour cells, e.g. myeloma cells) to produce hybridomas; and
(e) selectively culturing the hybridomas on the basis of the selectable phenotype conferred by the genetic material contained in the lymphocytes.

18. A monoclonal antibody producible by the method of claim 17 for use e.g. in therapy.

19. A method of selectively eliminating or depleting a particular tissue or cell type in an organism, comprising the steps of:
(a) providing a transgenic organism (e.g. a transgenic organism according to any one of claims 1 to 10) having a negative selectable marker operably linked to an expression element (e.g. a promoter) specific for the tissue or cell type to be eliminated or depleted;
(b) administering a selective agent to the organism to eliminate or deplete that tissue or cell type on the basis of the expression therein of the negative selectable marker.

20. A method according to claim 19 for modelling cell/tissue loss or atrophy (e.g. immunodegenerative or neurodegenerative diseases/disorders), wherein the tissue or cell type to be eliminated or depleted is that tissue or cell type which is subject to disease/disorder-related elimination or atrophy.

21. A transgenic organism according to any one of claims 1 to 10 for use in the method of claim 19 or claim 20.

22. An organism (for example a vertebrate, e.g. a mammal) in which a particular cell/tissue is specifically eliminated or depleted, produced by the method of claim 19 or claim 20.

23. An organism according to claim 22 which is a model of disease/disorder-related cell/tissue loss or atrophy, e.g. being a model of immunodegenerative or neurodegenerative diseases/disorders.

24. A method of screening compounds for pharmacological activity against a disease or disorder involving cell/tissue loss or atrophy (e.g. neurone loss and/or atrophy), comprising the steps of:
(a) providing a test model of the disease according to the method of claim 20;
(b) administering the compound to be tested to the test model of step (a);
(c) screening the compound to be tested on the basis of its effect on the test model.

25. A method according to claim 20 or claim 24 wherein the disease is:
(a) Parkinson's disease and the tissue or cell-type to be eliminated or depleted comprises dopaminergic neurones in the substantia nigra;
(b) Huntington's chorea and the tissue or cell-type to be eliminated or depleted comprises neural cells of the striatum;
(c) Alzheimer's disease and the tissue or cell-type to be eliminated or depleted comprises acetylcholine-, serotonin- and/or noradrenaline-neurones neurones associated with the neo- and palaeocortex;
(d) multiple sclerosis and the tissue or cell-type to be eliminated or depleted comprises brain oligodendrocytes;
(e) immune disease and the cell-type to be eliminated or depleted comprises CD3, CD4 and/or CD8 cells; and
(f) AIDS and the cell-type to be eliminated or depleted comprises CD4 cells.

26. A method (e.g. an in vitro method) of determining the effect of a deficit in a first class of cells (e.g. brain cells) on the characteristics of a second class of cells in an organism, the method comprising the steps of:
(a) providing a transgenic organism (for example an organism according to any one of claims 1 to 10) having a first negative selectable marker operably linked to an expression element specific for the first class of cells and either; (i) a positive selectable marker operably linked to an expression element specific for the second class of cells, or (ii) a second negative selectable marker linked to an expression element which directs the expression of the negative selectable marker in all cells of the organism except the second class of cells;
(b) administering a selective agent to the organism to induce a deficit in the first class of cells on the basis of the expression therein of the negative selectable marker;
(c) removing cells from the organism; and
(d) selectively culturing cells of the second class from those cells removed in stem (c) on the basis of; (i) the expression therein of the positive selectable marker, or (ii) the lack of expression therein of the negative selectable marker.
